(19) United States
(12) Patent Application Publication
(10) Pub. No.: US 2004/0146479 A1
(43) Pub. Date: Jul. 29, 2004

(54) Infection control system

(76) Inventor: Steven Kritzler, Cronulla (AU)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(21) Appl. No.: 10/481,992

(22) PCT Filed: Jul. 9, 2002
(86) PCT No.: PCT/AU02/00927

(30) Foreign Application Priority Data
Jul. 9, 2001 (AU) PR 6223

(51) Int. Cl. A61K 7/075; A61K 7/08

(52) U.S. Cl. 424/70.27

(57) ABSTRACT

A method for control of transmission of pathogenic organisms between a carer (of plurality of carers) and a patient (or plurality of patients) during a shift which includes the steps of (1) washing the hands of the carer, after commencement of a shift and prior to contact with the patient, with a first composition which assists in removal from the carer's hands of any anionic species of a kind which reduce the bactericidal efficacy of biocides, and (2) ensuring that no composition containing an anionic surfactant contacts the skin after step (1) and prior the end of the shift. A kit comprising a first composition in combination with a second composition, said compositions being such that the second contains a biocide, and use of the first prior to use of the second conditions the skin of a user against deactivation of the biocide of the second.

### TECHNICAL FIELD

This invention relates to an Infection Control System for use in hospitals, clinics, surgeries, and other locations at which it is important to eliminate, or at least minimize, the risk of infection and of spread of infection.

### BACKGROUND ART

The invention is herein described with reference to its applicability to hospitals but is not limited to use in that environment.

Modern antisepsis began as recently as the 1840's when Dr Holmes and Dr. Semmelweiss called attention to the contagiousness of puerperal fever (infection following childbirth). The former recommended cleanliness and the latter recommended the use of solutions of chloride of lime for washing the hands of attending physicians to destroy “cadaveric poisons”. By 1865 carbolic acid (phenol) was being widely used as an antiseptic.

It is now well understood that infection may be spread via skin contact through the transmission of pathogenic organisms. In hospitals, hand washing is now generally considered to be the most important measure in preventing spread of infection. Hand-washing protocols occupy a central role in the strategy for prevention of spread of infection. While in former times hands were merely washed with soap and water and/or lime chloride, this was later replaced with the practice of scrubbing the skin with a solution containing a surfactant followed by application of a disinfectant. The terms “disinfectant”, “biocide”, and “antimicrobial” are herein used interchangeably and in a general sense to include any substance which will kill or prevent the growth of micro-organisms and includes germicides, bactericides, bacteriostats, and if the context admits, fungicides, fungistats, sporicides and sporistats.

In recent years there have been developed sophisticated compositions for combining washing and disinfection in a single operation. Some of these compositions are aqueous antiseptic cleaning compositions, while others are non-aqueous (for example compositions having in excess of about 55% by weight of the composition of an alcohol). Both aqueous and non-aqueous types typically include an antimicrobial agent and one or a mixture of surfactants which may be anionic, cationic or nonionic surfactants.

It is known to formulators that some antimicrobial agents are incompatible with some surfactants. For example, chlorhexidine [N,N′-bis(4-chlorophenyl)-3,12-diimino 2,4,11,13-tetraazatetradecanediimide]digluconate or other soluble salts thereof are effective antimicrobial agents but are incompatible with, and are deactivated by, anionic surfactants and are reduced in activity by most non-ionic surfactants. Formulations combining the biocide with a non ionic such as polyoxyethylenepolyoxypropylene block copolymers require high amounts of antimicrobial to retain biocidal activity (which results in a high incidence of skin irritation) and high concentrations (20%-25%) of surfactant to maintain sudsing and skin cleansing effectiveness, are costly, and adversely de-fat skin with repeated application. Other antimicrobial agents are known to be incompatible with, and deactivated by, other surfactants. However a proliferation of aqueous and non-aqueous products comprising various combinations of one or more surfactant are marketed and one or more biocidal hand-wash preparations can now be found on or near wash-basins in most hospitals.

To assist in explaining the invention the term “carer” is herein used to describe “health-care personnel” including a person such as a doctor, nurse or other carer who is a potential carrier of pathogenic organisms from one patient to another. The “carer” will normally come into contact with a plurality of patients during a work “shift” As herein used “shift” means a period commencing from when the carer enters an infection controlled environment and terminating when the carer leaves that environment. For example a shift may commence or terminate with a tea break a lunch break or a visit to a toilet. The term “carer” may extend to include patients who are themselves subjected to an infection control protocol. During a shift a carer will follow an infection control protocol which defines when and how the carer washes his/her hands. Typically such protocols require that hands be washed before significant contact with any patient, or different sites on any patient, and after activities likely to cause contamination. Significant patient contact may include:

physical examination of a patient,

emptying a drainage reservoir (e.g. catheter bag),

undertaking venipuncture or delivery of injection,

changing any wound dressing,

or the like

Activities which can cause contamination include:

Handling equipment/instruments soiled with blood or other body substances,

direct contact with body secretions or excretions,

going to the toilet,

or the like

A typical aqueous hand-wash/hand-rub protocol, for example, involves wetting hands and wrists, applying 3 ml of the reference formulation to cupped hands, and using the following 6 steps each consisting of five strokes backwards and forwards:

Step 1 Palm to palm.

Step 2 Right palm over left dorsum and left palm over right dorsum.

Step 3 Palm to palm with fingers interlaced.

Step 4 Backs of fingers to opposing palms with fingers interlocked.

Step 5 Rotational rubbing of right thumb clasped in left palm and vice versa.

Step 6 Rotational rubbing backwards and forwards with clasped fingers of right hand in left palm and vice versa.

A protocol of this type requires approx 60 seconds of hand rubbing.

The protocol will also normally define hand drying techniques and specify hand-washing facilities such as type of basin, type of tap (foot or elbow controlled) etc. to be used

The infection control protocol in an infants ward, for example, typically requires a carer to wash his/her hands using an antimicrobial hand-wash after each occasion on which a baby is handled and before another is handled. It is not uncommon for a nurse to perform 140 or more hand-washing procedures per day. The high frequency of use of antimicrobial hand-wash agents tends to result in de-fatting of the skin and causes dermatological problems such as dry, cracked, or chaffed skin. Under existing hand-washing protocols, nurses suffering from cracked skin are required to be transferred to other duties to avoid infection or cross-infection. At many hospitals up to 20% of the nursing staff are assigned to other duties at any one time for this reason. That represents a major loss of skilled person-hours and a major community health cost.

Some nurses ignore the rules about scrubbing between each patient contact in an attempt to reduce skin damage. Recent studies have attributed significant rises in infection in a hospital wards to non-compliance. A recent review (D. Pittet and J. M. Boyce; The Lancet Infectious diseases April 2001) found a high rate of non compliance and recommended hand-washing with plain soap and water followed by use of an antiseptic agent, preferably an alcoholic hand rub, and other improvements in infection control practices. Some hospitals provide barrier creams and/or moisturizing creams to assist in reducing moisture removal or re-moisturizing of the skin after washing and a plethora of compositions are marketed for such purposes. Some nurses do not like the preparations provided by the hospital and use self-purchased compositions preferring the perfume, feel, or other quality of the self-purchased product.

The present inventor recently investigated a number of incidents in which cross-infection rates have risen to unacceptable levels in various hospital wards across the nation, notwithstanding the adoption of rigorous infection control protocols which required hand scrubbing with commercially available antibacterial hand-washing preparations before each patient contact. At first it was thought that the staff were ignoring the specified procedures, but on investigation, it was found that the problem persisted notwithstanding that the procedures were being strictly adhered to. The present invention arose from that investigation and resulted in an immediate and significant reduction in cross infection.

Any discussion of the prior art throughout the specification should in no way be considered as an admission that such prior art is widely known or forms part of common general knowledge in the field.

The object of the invention is to provide an infection control system which avoids or minimizes at least one or more of the abovementioned disadvantages of the prior art. The object of a preferred embodiment of the invention is to reduce or minimize the risk of infection transmission via skin contact. It is a further object to provide a set or kit of compositions suitable for putting the invention into practice.

### DESCRIPTION OF INVENTION

According to one aspect the invention provides a method for control of transmission of pathogenic organisms between a carer and a patient during a shift which includes the steps of:

(1) washing the hands of the carer, after commencement of a shift and prior to contact with the patient, with a first composition which assists in removal from the carer's hands of any anionic species of a kind which reduces the bactericidal efficacy of biocides, and

(2) ensuring that no composition containing an anionic surfactant contacts the skin after step (1) and prior the end of the shift.

The first composition may, but preferably does not, contain a biocide.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”.

According to a second aspect the invention provides a method for control of transmission of pathogenic organisms between a plurality of carers and one or more patients during a shift which includes the steps of:

(1) washing the hands of each carer, after commencement of a shift and prior to contact with any patient, with a first composition which assists in removal from the carer's hands of any anionic species of a kind which reduce the bactericidal efficacy of biocides, and

(2) ensuring that no composition containing an anionic surfactant contacts any carer's skin after step (1) and prior the end of the shift.

In preferred embodiments of the invention during the shift, after step 1, and prior to significant contact with a patient, each carer will wash hands with a second composition comprising one or more detergents and one or more biocides, the second composition excluding any anionic surfactant, and will ensure that no composition containing an anionic surfactant contacts the skin after step (1) and before the end of the shift

In preferred embodiments of the invention the hands of the carer will be washed with the second composition prior to contact with each successive patient and after any activities likely to cause contamination. The second composition may for example be an aqueous or non aqueous (e.g. alcoholic) biocidal composition such as an antiseptic hand-ash, surgical scrub, or antiseptic hand rub or may be a moisturising lotion, hand-cream, or the like. Typically a number of such second compositions will be used during a shift and according to the invention these must each be free of anionic surfactants.

Preferred embodiments of a method according to the invention further comprises the step of applying a third composition which is a barrier cream or a hand cream which is devoid of any anionic surfactant to the hands of the carer at a time during a shift after step (1).

The invention also provides a kit comprising a first composition in combination with a second composition, said compositions being such that use of the first conditions the skin of a user against deactivation of the biocide of the second.

The invention provides a combination of compatible compositions comprising a first hand-wash effective to remove anionic species of a kind which deactivate biocides from a carer's hands, and a compatible biocidal hand-wash of which the biocidal efficacy is not reduced on skin washed or preconditioned with the first hand-wash.

In preferred embodiments the first hand-wash does not contain a biocidal active. In a highly preferred embodiment the combination further includes a compatible hand-cream use of which does not significantly reduce the biocidal efficacy of the biocidal hand-wash on skin washed with the first hand-wash.

In studying cross infection in hospital wards the present inventors were surprised to discover that various brands of antiseptic hand wash in use in the hospital, and all of which produced consistently effective bactericidal properties when tested in the laboratory, nevertheless failed to work effectively in the workplace. It was then found that the effectiveness varied from user to user and day to day and eventually it was discovered that the user's skin could in many cases destroy or greatly reduce the antiseptic effect of the biocide, rendering scrubbing with an antiseptic hand-wash largely ineffective and giving rise to a statistically significant climb in cross-infection rates.

This unpredicted variation was eventually traced to anionic surfactants which had become absorbed on the carer's hands from products such as dishwasher and laundry detergents used at home, self purchased hand creams, shampoos, shaving soaps and cosmetics used before the commencement of a shift. These products left traces of anionic surfactant on or in the skin of the Carer's hands. It is not clear whether these traces are present as an invisible film on, adsorbed on, absorbed on, or complexed with the skin. Surprisingly, these invisible traces of anionic surfactant were sufficient to significantly reduce the biocidal efficacy of incompatible biocidal hand wash products and remained on the skin even after repeated hand-washing. In addition it was found that in many hospitals a choice of antiseptic hand wash preparations was available. Sometimes two brands were available at one sink. In other cases one brand would be available at one sink and a different brand at another sink in the same ward. It was found that these differing brands often interacted so that one would inactivate the antiseptic component of the other. This was because one would contain an anionic detergent and a compatible biocide in sufficient concentration to present activity in the presence of the anionic, while the other would contain a biocide that was deactivated by the anionic surfactant. A nurse would scrub at one sink using one brand of hand wash preparation, and then subsequently use a different brand preparation at the same or another sink. The second brand would be deactivated by virtue that the nurses hand's still retained residues from the previous brand (incompatible) hand wash preparation. Sometimes also, the hand creams provided by the hospital and/or those self-purchased by the carer contained anionic surfactants which became substantive to the skin and destroyed the efficacy of the antiseptic hand-wash. While existing hand-wash protocols provided against skin infection by organisms, they did not address skin contamination by surfactants.

While it has been known to formulators that certain biocides were deactivated by certain surfactants, this was not generally known among users. It has not previously been appreciated that the efficacy of a biocidal hand wash was significantly affected by surfactant species adsorbed on or in the skin.

Thus while infection control protocols have focused on bactericidal efficacy, they have failed to take account of the condition of the carer's hands prior to washing with biocidal hand wash preparations and the effect of this on the efficacy of the hand-wash.

It was also known that some antimicrobials could be adsorbed and retained on skin. Thus for example ASTM E1174-94 (a Standard Test Method for Evaluation of Health Care Personnel Hand-wash) Para 8.2 specifies that panellists “avoid contact with antimicrobials (other than the test formulation) for the duration of the test and for at least one week prior”. However there is no precaution in respect of prior use of surfactants by the panellists. It has not hitherto been appreciated either (1) that serial use of incompatible antiseptic hand-washes can nullify infection control protocols or (2) that surfactants absorbed and retained on skin can seriously prejudice the efficacy of such protocols and that these are significant causes of cross-infection.

The present inventor found that such cross-infection could be prevented by the method of the invention.

The first step of the method of the invention involves washing the hands of each carer, after commencement of a shift and prior to contact with any infected person, with a first composition (“pre-shift wash”) which excludes any anionic surfactant, and which assists in removal of any anionic species such as an anionic surfactant absorbed on the carer's hands, from use prior to the shift of hand soaps, dishwasher or laundry detergents, hand creams, shampoos, cosmetics or other sources A preferred embodiment of such a composition is described hereinafter in example 1.

“Removal” in this context includes complexing, neutralizing or other means for deactivating an anionic species present, as well as simple physical removal, which is preferred. This first step removes any anionic surfactant which has become absorbed on the carer's hands but in less preferred embodiments any anionic surfactant in the skin is complexed in such a way as to prevent subsequent interaction with the second composition without necessarily removing it from the skin. It is an important step in the protocol that this step be performed at the commencement of each work shift. Use of the pre-shift wash which is devoid of any anionic surfactant but which in the preferred embodiment contains non ionic surfactants, amphoteric surfactants, and water conditioners, is effective to remove any anionic surfactants which may be adsorbed on or in the carer's skin. There is no need for this first composition to contain a biocide since the purpose of the first step is removal of cationic surfactants not control of infection. However if desired a compatible biocide could be included.

The Carer will next normally perform an antiseptic hand-wash using the second composition which does contain a biocide and may thereafter apply a hand-cream using a third composition which is devoid of any anionic detergent and which is compatible with the first and second compositions.

The antiseptic hand-wash will be repeated before each new patient is handled or otherwise as necessary utilizing the second composition (i.e. a compatible biocidal hand wash or hand rub or surgical scrub). However the first composition need not be used again during the shift as defined. However if the carer leaves the disinfection controlled area, for example for a tea break, then the first step would be repeated before the first antiseptic hand-wash in case the hands had become contaminated with an anionic surfactant prior to re-entry of the controlled area.

The first composition wash, the antiseptic hand-wash and the hand-cream are desirably made available as a kit of inter-compatible disinfection prevention components in appropriate volumetric ratios of each to the others. While the first composition need not contain any biocide, and of itself need not be effective as an antiseptic, its use is essential to prevent the antiseptic hand-wash from being rendered ineffective.

### BEST MODES FOR CARRYING OUT THE INVENTION

The invention will now be more particularly described by way of example only with reference to specific formulations.

### TESTS OF COMPATIBILITY OF PRIOR ART

A study of compatibility of a major brand biocidal hand-wash and other products commonly used in leading Australian hospitals was undertaken and compared with compatibility of compositions according to the invention.

The following products were obtained from Regional Healthcare Pty Ltd.

Microshield¹ 2% ¹ Microshield is a registered trademark of Johnson and Johnson Inc.

Microshield 4%

Microshield PVP

Microshield T

Microshield Hand rub

Hospital Skin Care Lotion (Smith & Nephew).

### METHODOLOGY OF COMPATIBILITY TESTS

Each compatibility test was carried out using the following procedure:

(i) The CHG product and the Triclosan product or PVP/I were intimately mixed in a ratio of 10:3 with a magnetic stirrer. For CHG product lotions and alcoholic CHG products and Triclosan the mixing ratio was 1:1).

(ii) The mixture was allowed to stand for 5 minutes (Sample 1) and overnight (Sample 2).

(iii) The samplers were mixed with water at a ratio of 1:10, the dilution was mixed by vortex and centrifuges.

(iv) The supernatant was taken for analysis by HPLC using the eluant to dilute the sample by 20 mL.

The HPLC conditions were as follows:

| | |
|---|---|
| Mobile phase | 65% Methanol in water with 0.57 g sodium acetate |
| | (anh), 1 g heptanesulphonic acid buffered to pH |
| | 4.0 with glacial acetic acid. |
| Pump flow rate | Isocratic @ 1.10 mL/min. |
| Column | 3.9 × 300 mm Novapak C₁₈ reverse phase ODS |
| Column temperature | 45.0° C. |
| Injection volume | μL |
| Detector | UV @ 258 nm. |

A calibration graph of peak area versus standard chlorhexidine was prepared with each sample batch.

The calculation of “decrease in CHG content” of the sample was based on comparison of the CHG in the initial sample with the sample after mixing with the second component at the same dilution.

### RESULTS OF TEST OF COMPATIBILITY OF PRIOR ART

The results of the analyses expressed as a percentage decrease in chlorhexidine content in mixtures of products A and B were as shown in Table

TABLE 1

% Decrease in CHG

| Product A | Product B | Immediate | Overnight |
|---|---|---|---|
| Microshield 2% | Microshield T | 88% | 78% |
| Microshield 4% | Microshield T | 94% | 97% |
| Microshield 2% | Microshield | 97% | 96% |
| | PVP | | |
| Microshield 4% | Microshield | 11% | 11% |
| | PVP | | |
| Microshield 2 | Microshield | 57% | 57% |
| | Moisturising Ltn. | | |
| Microshield 4% | Microshield | 22% | 22% |
| | Moisturising Ltn | | |

As is apparent the immediate % decrease ranges from 11% to 97% of chlorhexidine with potentially serious implications for infection control.

The results show that there is an incompatibility when these products are combined so that if residues of one antiseptic hand-wash or rub are left on the skin of a carer as a result of incomplete rinsing, and another is subsequently used then it would be expected from these results that the in vivo efficacy of these products would be significantly compromised by their interaction.

Similar results were obtained when incompatible products from different manufacturers were used serially during a shift.

It has also been found that when hands are washed with one antiseptic hand-wash, then washed with ordinary soap or liquid soap (both of which contain anionic species) and then washed with a second hand-wash compatible with the first antiseptic hand-wash then the biocidal efficacy of the second hand-wash is greatly reduced. That reduction does not occur when the first preparation according to the invention (example 1) is used in place of the soap.

### RESULTS OF TEST OF COMPATIBILITY OF EXAMPLES ACCORDING TO INVENTION

The experiment was repeated using products according to the invention. In all cases the % decrease (immediate and overnight) was less than 10% and in most cases less than 5% The results for selected combinations are shown in Table 2.

TABLE 2

% Decrease in CHG

| Product A | Product B | Immediate | Overnight |
|---|---|---|---|
| Example 1 | Example 2 | 0% | 0% |
| Example 1 | Example 3 | 0% | 0% |
| Example 1 | Example 4 | 0% | 0% |
| Example 1 | Example 5 | 0% | 0% |
| Example 1 | Example 6 | 0% | 0% |
| Example 1 | Example 7 | 0% | 0% |
| Example 1 | Example 8 | 0% | 0% |
| Example 1 | Example 9 | 0% | 0% |
| Example 2 | Example 3 | 8% | 8% |
| Example 2 | Example 4 | 8% | 8% |
| Example 2 | Example 5 | 0% | 0% |
| Example 2 | Example 6 | 0% | 0% |
| Example 2 | Example 7 | 0% | 0% |
| Example 2 | Example 8 | 0% | 0% |
| Example 2 | Example 9 | 0% | 0% |
| Example 3 | Example 4 | 2% | 2% |
| Example 3 | Example 5 | 3% | 2% |
| Example 3 | Example 6 | 4% | 4% |
| Example 4 | Example 6 | 0% | 0% |
| Example 4 | Example 5 | 4% | 0% |
| Example 4 | Example 6 | 0% | 0% |
| Example 5 | Example 4 | 4% | 0% |
| Example 5 | Example 9 | 4% | 3% |
| Example 5 | Example 6 | 7% | 3% |
| Example 7 | Example 4 | 6% | 8% |

The binary combinations not shown in table 2 each showed no detectable increase.

The results show that the products exemplified are compatible and that there is no, or minimal, loss in efficacy when the compositions are combined

### Example 1

### Pre-Shift Hand Wash

| Materials | Contents(% W/V) |
|---|---|
| Akylpolyglucoside (APG) | 4.0000 |
| Disodium Cocoamphodipropionate | 1.0000 |
| Cocamidpropyl Betaine | 1.0000 |
| Macrogol 400 | 2.0000 |
| Ethyleneglycolmonostearate | 1.0000 |
| Isothiazoline Derivatives | 0.0014 |
| Fragrance | 0.2000 |
| Water Purified | qs to 100% volume |

If desired pH can be adjusted to 6.5 with citric acid which has been found not to reduce the biocidal efficacy of CHG.

### Example 2

### Moisturising Hand Lotion

| Materials | Contents(% W/V) |
|---|---|
| Mineral Oil | 3.00 |
| Isopropylmyristate(IPM) | 1.00 |
| Cetostearyl Alcohol | 2.50 |
| Polyethylene Glycol 400O | 2.30 |
| Polysorbate 60 | 1.00 |
| Glyceryl Stearate | 0.50 |
| PEG-100 Stearate | 0.50 |
| Methyl Paraben | 0.20 |
| Propyl Paraben | 0.10 |
| Fragrance | 0.10 |
| Water Purified | qs to 100% volume |

### Example 3

### Antiseptic Hand Wash (2% Chq)

| Materials | Contents(% W/V) |
|---|---|
| Propan-1-ol | 2.00 |
| Hydroxy Ethyl Cellulose | 0.60 |
| Akylpolyglucoside(APG) | 4.00 |
| Lauramide Oxide | 1.50 |
| Cocamide DEA | 0.80 |
| Disodium Cocoamphodipropionate | 0.80 |
| Glycerol | 1.00 |
| Chlorhexidine Gluconate | 2.00 |
| Fragrance | 0.10 |
| Dyestuff (D&C Red 33) | 0.00040 |
| Lactic Acid | qs to pH 5.5 |
| Water Purified | qs to 100% volume |

### Example 4

### Antiseptic Hand Wash (1% Triclosan)

| Materials | Contents(% W/V) |
|---|---|
| Triclosan | 1.00 |
| Propan-1-ol | 5.00 |
| Propylene Glycol | 6.00 |
| Polyethylene Glycol 400 | 4.00 |
| Phenoxy Ethanol | 0.30 |
| Perfume | 0.20 |
| Akylpolyglucoside(APG) | 4.50 |
| Cocamidpropyl Betaine | 1.20 |
| Disodium Cocoamphodipropionate | 3.20 |
| Hydroxy Ethyl Cellulose | 0.55 |
| Edtate Sodium | 0.20 |
| Dyestuff (D&C Green No 3) | 0.00030 |
| Citric Acid(To pH 6.5) | 0.10 |
| Water Purified | qs to 100% volume |

### Example 5

### Surgical Scrub (4% CHG)

| Materials | Contents(% W/V) |
|---|---|
| Propan-1-ol | 2.00 |
| Hydroxy Ethyl Cellulose | 0.60 |
| Akylpolyglucoside(APG) | 4.00 |
| Lauramide Oxide | 1.60 |
| Cocamide DEA | 1.60 |
| Disodium Cocoamphodipropionate | 0.80 |
| Glycerol | 1.00 |
| Chlorhexidine Gluconate | 4.00 |
| Fragrance | 0.10 |
| Dyestuff(D&C Red 33) | 0.00040 |
| Lactic Acid | qs to pH 5.5 |
| Water Purified | qs to 100% volume |

### Example 6

### Surgical Scrub (0.75% Av. Iodine)

| Materials | Contents(% W/V) |
|---|---|
| Povidone | 2.25 |
| Iodine | 0.75 |
| Ethanol | 4.00 |
| Potassium Iodide | 1.30 |
| Propy-2-ol | 0.80 |
| Hydroxy Ethyl Cellulose | 0.60 |
| Propylene Glycol | 5.00 |
| Akylpolyglucoside(APG) | 3.60 |
| Sodium Nitrate | 0.55 |
| Triethanolamine | qs to pH 5.50 |
| Water Purified | qs to 100% volume |

### Example 7

### Alcoholic Hand Rub (0.5% Chg In 70% Ethanol)

| Materials | Contents(% W/V) |
|---|---|
| Ethanol (For 70% V/V) | 55.23 |
| Chlorhexidine Digluconate | 0.50 |
| Glycerol | 0.80 |
| Akylpolyglucoside(APG) | 2.10 |
| Benzyl Alcohol | 0.55 |
| Peg-75 Lanolin | 0.10 |
| Isopropylmyristate(IPM) | 0.05 |
| Fragrance | 0.10 |
| Dyestuff(Fd&C Red No 33) | 0.00020 |
| Lactic Acid (To pH 5.50) | 0.10 |
| Triethanolamine(To pH 5.50) | 0.10 |
| Water Purified | qs to 100% volume |

### Example 8

### Alcoholic Hand Rub (0.6% Triclosan)

| Materials | Contents(% W/W) |
|---|---|
| Ethanol (For 70% v/v) | 62.7600 |
| Triclosan | 0.5700 |
| Glycerol | 0.9100 |
| Akylpolyglucoside (APG) | 2.3900 |
| Phenoxyethanol | 0.6300 |
| Peg-75 Lanolin | 0.1100 |
| Isopropylmyristate (IPM) | 0.0570 |
| Fragrance | 0.1100 |
| Dyestuff (Fd&C Red No 33) | 0.0002 |
| PVP K30 | 0.1100 |
| Water | 32.3528 |
| Total | 100.0000 |

### Example 9

### Alcoholic Hand Rub (0.5% Triclosan/Povidone Iodide In 70% Ethanol

| Materials | Contents(% W/V) |
|---|---|
| Ethanol (For 70% v/v) | 55.23 |
| Triclosan | 0.50 |
| Propylene Glycol | 0.50 |
| Povidone Iodine | 4.00 |
| Potassium Iodide | 0.20 |
| Benzyl Alcohol | 0.35 |
| Peg-75 Lanolin | 0.25 |
| Polyethylene Glycol 400 | 6.00 |
| Polyethylene Glycol 4000 | 2.00 |
| Phosphoric Acid (To pH 5.50) | 0.10 |
| Triethanolamine(To pH 5.50) | 0.10 |
| Water Purified | qs to 100% |
| | volume |

As will be apparent to those skilled in the art from the teaching hereof the risk of cross infection can be significantly reduced by adopting a method of infection control according to the invention. The invention is not restricted to use of the formulations exemplified and can be conducted using other formulations in accordance with the teaching herein contained without departing from the inventive concept disclosed above.

1. A method for control of transmission of pathogenic organisms between a carer and a patient during a shift which includes the steps of:
(1) washing the hands of the carer, after commencement of a shift and prior to contact with the patient, with a first composition which assists in removal from the carer's hands of any anionic species of a kind which reduce the bactericidal efficacy of biocides, and
(2) ensuring that no composition containing an anionic surfactant contacts the skin after step (1) and prior-the end of the shift.

2. A method for control of transmission of pathogenic organisms between a plurality of carers and one or more patients during a shift which includes in the steps of:
(1) washing the hands of each carer, after commencement of a shift and prior to contact with any patient, with a first composition which assists in removal from the carer's hands of any anionic species of a kind which reduces the bactericidal efficacy of biocides, and
(2) ensuring that no composition containing an anionic surfactant contacts any carer's skin after step (1) and prior the end of the shift.

3. A method according to claim 1 or claim 2 including the further step of washing said hands, during said shift, after step 1, and prior to significant contact with a patient, with a second composition comprising one or more detergents and one or more biocides, said second composition excluding any anionic surfactants.

4. A method according to any one of the preceding claims wherein the hands of a carer are washed, prior to contact with each successive patient and after any activity likely to cause contamination, with a second composition comprising one or more detergents and one or more biocides, said second composition excluding any anionic surfactant,

5. A method according to claim 4 wherein the second composition is an aqueous composition.

6. A method according to claim 4 or claim 5 wherein the second composition is selected from the group consisting of antiseptic hand-washes, surgical scrubs, antiseptic hand rubs, antiseptic creams, moisturising lotions, hand-creams, or the like.

7. A method according to any one of the preceding claims further comprising the step of applying a barrier cream or a hand cream which is devoid of any anionic surfactant to the hands of the carer at a time during a shift and after step (1).

8. A method according to any one of the preceding claims wherein the first composition comprises an alkylpolyglucoside surfactant and no anionic species.

9. A method according to any one of claims 1 to 7 wherein the first composition comprises an alkylpolyglucoside surfactant and one or more compatible anionic species which do not effect biocidal activity.

10. A method according to claim 9 wherein the composition contains an alkylpolyglucoside surfactant together with EDTA or citric acid.

11. A method of infection control comprising the step of (1) removing any anionic species from a carers hand at the start of a shift and prior to any patient contact, and (2) thereafter during the shift avoiding hand contact with any composition containing an anionic surfactant.

12. A kit when used in the method of any one of the preceding claims comprising a first composition in combination with a second composition, said compositions being such that the second contains a biocide, and use of the first prior to use of the second conditions the skin of a user against deactivation of the biocide of the second.

13. A kit according to claim 12 further including a compatible hand-cream.

14. A kit according to claim 12 or 13 wherein the first composition comprises an alkylpolyglucoside formulated hand-wash which excludes any anionic species of a kind which reduces the bactericidal efficacy of biocides.

15. A kit according to claim 12 or 13 wherein the first composition comprises an alkylpolyglucoside formulated hand-wash which excludes any anionic species of a kind which reduces the bactericidal efficacy of biocides.

16. A kit according to claim 15 wherein the first composition comprises an alkylpolyglucoside surfactant and excludes any anionic species.

17. A combination comprising a first hand-wash effective to remove anionic species absorbed on a carer's hands, and a compatible biocidal hand-wash of which the biocidal efficacy is not reduced on skin washed with said first hand-wash.

18. A combination according to claim 17 wherein the first hand-wash does not contain a biocidal active.

19. A combination according to claim 17 or 18 wherein the combination further includes a compatible hand-cream.

20. A combination according to any one of claims 17 to 18 wherein the first composition comprises an Alkylpolyglucoside formulated hand-wash which excludes any anionic species.

21. A method substantially as herein described.

22. A composition substantially as herein described with reference to any one of the examples.

23. A kit comprising a composition according to example 1 in combination with any one or more of the other compositions herein exemplified.
